(19) United States
(12) Patent Application Publication
(10) Pub. No.: US 2026/0117295 A1
(43) Pub. Date: Apr. 30, 2026

(54) CHROMATOGRAPHY-BASED MULTIPLEX NUCLEIC ACID DETECTOR, DETECTION TEST STRIP AND DETECTION METHOD

(71) Applicant: BIOTEKE CORPORATION (WUXI) CO., LTD., Jiangsu (CN)

(72) Inventors: Yunpeng Li, Jiangsu (CN); Yuxi Cai, Jiangsu (CN); Shanshan Song, Jiangsu (CN); Zhitu Zhou, Jiangsu (CN)

(21) Appl. No.: 18/681,835

(22) PCT Filed: Sep. 5, 2023
(86) PCT No.: PCT/CN2023/117047

§ 371 Date: Feb. 6, 2024

(30) Foreign Application Priority Data
Jun. 21, 2023 (CN) 202310747346.7

(51) Int. Cl. C12Q 1/686; B01L 7/00; C12Q 1/34; C12Q 1/48; C12Q 1/6876

(52) U.S. Cl. CPC C12Q 1/686; B01L 7/52; C12Q 1/34; C12Q 1/48; C12Q 1/6876; C12Y 207/07049; C12Y 302/02027; B01L 2300/04; B01L 2300/0825; B01L 2300/1805; C12Q 2600/16

(57) ABSTRACT

A chromatography-based multiplex nucleic acid detector includes an air supply device, a heating device, a polymerase chain reaction (PCR) pipeline, and a cover plate assembly. An air outlet of the air supply device is configured to communicate with a first end of an amplification reagent tube. The cover plate assembly is mounted on the heating device, where an observation window is formed on the cover plate assembly, a PCR pipeline installation position is formed on at least one of the cover plate assembly or the heating device, the PCR pipeline is configured to be mounted at the PCR pipeline installation position, the heating device is configured to heat the PCR pipeline, and a test strip is mounted at the observation window. A chromatography-based multiplex nucleic acid detection test strip and a chromatography-based multiplex nucleic acid detection method are further provided.

This application claims priority to Chinese Patent Application No. 202310747346.7 filed on Jun. 21, 2023, the disclosure of which is incorporated herein by reference in its entirety.

### TECHNICAL FIELD

The present application relates to the field of biological detection, for example, a chromatography-based multiplex nucleic acid detector and detection test strip.

### BACKGROUND

Currently, nucleic acid detection is widely used in various fields such as criminal investigation, pathogenic microorganism detection, and disease diagnosis. Generally, the target nucleic acid content in an environmental sample or a physiological sample is very low To make the result more accurate, the target nucleic acid fragment needs to be amplified. The polymerase chain reaction (PCR) is the most widely used nucleic acid amplification technology. The amplification product obtained by the PCR technology is determined to be negative or positive according to the band size through agarose gel electrophoresis. The operation is cumbersome, time-consuming, prone to contamination, and has low specificity. In the related art, detection may be performed in a fluorescence quantitative PCR method, but due to the impact of a detection channel, the number of detection targets in a single tube is limited. At the same time, the PCR technology in the related art needs to be adopted with a PCR instrument, and common 200 μL eight-tube consumables are mostly used for amplification and optical detection, resulting in high detection costs.

Chromatography technology is a rapid diagnostic technology that uses colloidal gold and other chromogenic particles as tracers for antigen-antibody reactions and has the advantages of being fast, simple, and economical without any special equipment. However, due to relatively low detection sensitivity, weak specificity, and a narrow scope of application, chromatography technology is mainly suitable for rapid preliminary screening of protein levels.

In the related art, the PCR detection has relatively strong specificity and high sensitivity, and the chromatographic reaction is relatively fast and simple. Therefore, there is an urgent need for a detector to combine the advantages of both the PCR detection and the chromatographic reaction for screening the nucleic acid.

### SUMMARY

The present application proposes a chromatography-based multiplex nucleic acid detector that can screen the nucleic acid in conjunction with the advantages of strong specificity and high sensitivity of PCR detection and a fast and simple chromatographic reaction.

The present application proposes a chromatography-based multiplex nucleic acid detector. The chromatography-based multiplex nucleic acid detector includes an air supply device, a heating device, a PCR pipeline, and a cover plate assembly. An air outlet of the air supply device is configured to communicate with a first end of an amplification reagent tube. The cover plate assembly is mounted on the heating device. An observation window is formed on the cover plate assembly. A PCR pipeline installation position is formed on the cover plate assembly and/or the heating device, and the PCR pipeline is configured to be mounted at the PCR pipeline installation position. The heating device is configured to heat the PCR pipeline, and the observation window is used for mounting a test strip. A second end of the amplification reagent tube communicates with a first end of the PCR pipeline, and a second end of the PCR pipeline is configured to output an amplified sample to the test strip.

The present application further proposes a chromatography-based multiplex nucleic acid detection test strip applied to the preceding multiplex nucleic acid detector.

The present application further proposes a chromatography-based multiplex nucleic acid detection method being applied to the preceding multiplex nucleic acid detection test strip and the preceding multiplex nucleic acid detector, and the method includes the steps described below.

A to-be-tested nucleic acid sample is added to a multiplex PCR amplification reagent set.

Temperature-changing amplification is performed on the multiplex PCR amplification reagent set.

An amplification product is detected by the test strip and a result is read from the test strip.

### BRIEF DESCRIPTION OF DRAWINGS

FIG. 1 is a schematic view of the overall structure of a multiplex nucleic acid detector according to the present application;

FIG. 2 is a schematic view illustrating the connection between an amplification reagent tube and an air pipe according to the present application;

FIG. 3 is an exploded view of a multiplex nucleic acid detector according to the present application;

FIG. 4 is a structural view of a PCR pipeline according to the present application;

FIG. 5 is a structural view of a base plate and a heating assembly according to the present application;

FIG. 6 is an enlarged view of part A in FIG. 5;

FIG. 7 is a schematic view illustrating the assembly of a test strip according to the present application;

FIG. 8 is a structural view of a test strip according to the present application;

FIG. 9 is a view illustrating a chromatography detection result of multiplex gene amplification of the negative and single-target DNA/RNA for RSV, ADV, HPIV1, MP, and HRV;

FIG. 10 is a view illustrating a chromatography detection result of multiplex gene amplification of the dual-target DNA/RNA for RSV, ADV, HPIV1, MP, and HRV;

FIG. 11 is a view illustrating a chromatography detection result of the multiplex gene amplification of the triple-target DNA/RNA for RSV, ADV, HPIV1, MP, and HRV;

FIG. 12 is a view illustrating chromatography detection results of multiplex gene amplification of the quadruple-target DNA/RNA and quintuple-target DNA/RNA for RSV, ADV, HPIV1, MP, and HRV;

FIG. 13 is a view illustrating chromatography detection sensitivity result of multiplex gene amplification of the target DNA/RNA for RSV, ADV, HPIV1, MP, and HRV; and

FIG. 14 is a schematic diagram of an air pump according to an embodiment.

LIST OF REFERENCE NUMERALS

| Reference | | Reference | |
|---|---|---|---|
| numeral | Name | numeral | Name |
| 100 | Body | 200 | Amplification |
| | | | reagent tube |
| 210 | Liquid outlet | 300 | Air supply device |
| 310 | Air pump | 320 | Air pipe |
| 330 | Air pipe interface | 410 | Cover plate assembly |
| | cover | | |
| 411 | Cover plate | 412 | Window groove |
| 413 | Test strip slot | 414 | Test tube slot |
| 415 | Flume tank | 416 | First snap group |
| 417 | Second snap groove | 420 | Heating device |
| 421 | Base plate | 422 | High-temperature |
| | | | region |
| 423 | Constant-temperature | 424 | Low-temperature |
| | region | | region |
| 425 | Limiting block | 426 | T-slot |
| 427 | Slot opening | 430 | Heating assembly |
| 431 | Low-temperature | 432 | High-temperature |
| | part | | part |
| 433 | Constant-temperature | 440 | PCR pipeline |
| | part | | |
| 500 | Test strip | 510 | Sample pad |
| 520 | Marker pad | 530 | Capture membrane |
| 540 | Water absorbent pad | 550 | Support base plate |
| 560 | Line C | 570 | Line T |
| 600 | Observation window | | |

### DETAILED DESCRIPTION

If a directional indication is involved in the embodiments of the present application (for example, up, down, left, right, front, rear, or the like), the directional indication is merely used for explaining the relative positional relationship, motion, and the like between the components in a particular posture (as shown in the drawings). If the particular posture changes, the directional indication changes accordingly.

Moreover, terms such as “first” and “second”, if used herein, are for description only and are not to be construed as indicating or implying relative importance or implicitly indicating the number of technical features as indicated. Therefore, a feature defined as a “first” feature or a “second” feature may expressly or implicitly include at least one of the feature. In addition, “and/or” in the full text means including three parallel schemes. For example, “A and/or B” means including scheme A, scheme B, or a scheme that satisfies both A and B at the same time. In addition, the technical schemes of the embodiments can be combined with each other, but the combination must be capable of being achieved by those of ordinary skill in the art. When the combination of the technical schemes is contradictory or impossible to implement, such a combination does not exist and is not within the scope of the present application.

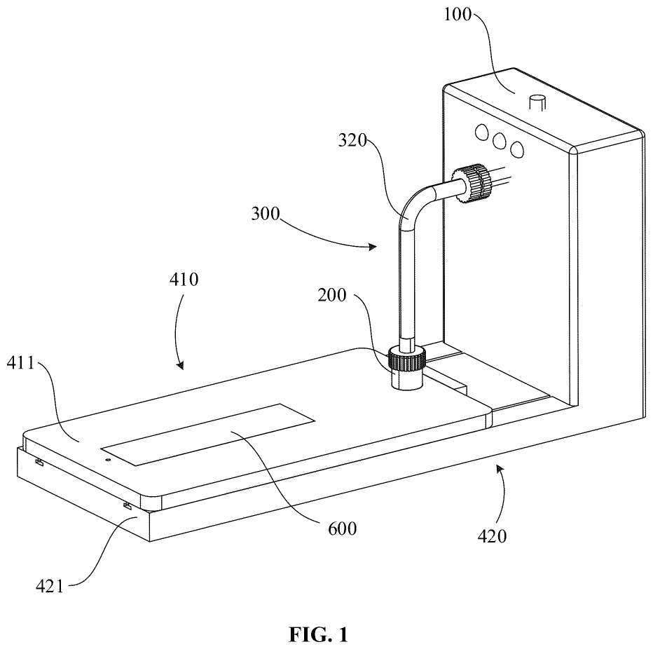

Referring to FIG. 1, the present application proposes a chromatography-based multiplex nucleic acid detector that can quickly perform multiplex nucleic acid screening on samples so that the sensitivity and accuracy of the screening can be ensured, at the same time, the nucleic acid screening efficiency can be improved. The multiplex nucleic acid detector proposed in the present application includes the following parts: a body 100, a heating device 420, an air supply device 300, and a cover plate assembly 410. An amplification reagent tube 200 contains a to-be-tested nucleic acid sample. The heating device 420 is disposed on a side of the body 100, and the heating device 420 heats a PCR pipeline 440, that is, the heating device 420 heats the to-be-tested nucleic acid sample to achieve the temperature condition for the nucleic acid amplification. The cover plate assembly 410 fixes the PCR pipeline 440 on the heating device 420. The air supply device 300 is disposed inside the body 100. The air supply device 300 pushes the to-be-tested nucleic acid sample in the amplification reagent tube 200 into the PCR pipeline 440 to complete the amplification of the to-be-tested nucleic acid sample. A test strip 500 is a chromatography test strip. The test strip 500 performs detection on the amplified to-be-tested nucleic acid sample and converts the detection into a visualized nucleic acid detection result. In an example, a PCR pipeline installation position is formed on at least one of the cover plate assembly 410 or the heating device 420, and the PCR pipeline 440 is configured to be mounted at the PCR pipeline installation position.

In the technical schemes of the embodiments of the present application, through the cooperation between the heating device 420 and the air supply device 300, the to-be-tested nucleic acid sample that enters the PCR pipeline 440 is rapidly amplified, and the test strip 500 directly interprets the amplified to-be-tested nucleic acid sample so that the chromatography-based multiplex nucleic acid detector provided in the present application can combine the advantages of PCR detection and chromatography to rapidly and accurately interpret the nucleic acid. In addition, the test strip is used for interpretation, that is, the visualization of a genetic detection result is achieved, and compared with the PCR amplification detection in the related art, the result interpretation of the test strip 500 is simpler and more immediate, thereby improving the detection efficiency; and the detection process does not require expensive professional equipment, thereby reducing detection costs. Furthermore, a chromatography-based multiplex nucleic acid detection method provided in the present application is applied to the preceding multiplex nucleic acid detector and detection test strip. In the multiplex nucleic acid detection method provided in the present application, while the accuracy of the nucleic acid detection result is ensured, the multiplex nucleic acid detection is achieved, that is, rapid screening in one sampling is achieved, and the simultaneous amplification of up to 13 target sequences is achieved, thereby improving the detection efficiency.

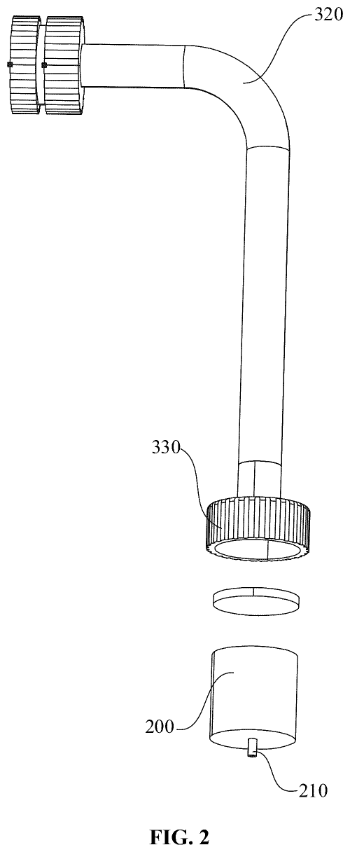

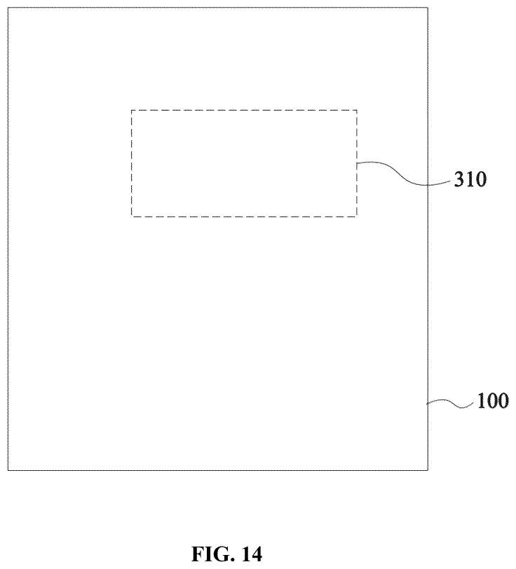

Referring to FIG. 1, FIG. 2, and FIG. 14, in an embodiment of the present application, to allow the to-be-tested nucleic acid sample to enter the PCR pipeline 440 from the amplification reagent tube 200, a liquid outlet 210 is disposed at the bottom of the amplification reagent tube 200 and connected to the PCR pipeline 440 so that the amplification reagent containing the to-be-tested nucleic acid sample directly enters the PCR pipeline 440 from the liquid outlet 210. To push the amplification reagent containing the to-be-tested nucleic acid sample from the amplification reagent tube 200, the air supply device 300 in the body 100 includes an air pump 310 and an air pipe 320. The air pump 310 is disposed inside the body 100. Two ends of the air pipe 320 are separately connected to an air outlet of the air pump 310 and the top of the amplification reagent tube 200 so that when the air pump 310 is working, airflow enters the amplification reagent tube 200 through the air pipe 320, the air pressure in the amplification reagent tube 200 increases, and then the amplification reagent containing the to-be-tested nucleic acid sample is pushed into the PCR pipeline 440. Moreover, the air pump 310 is used so that the air pressure in the amplification reagent tube 200 is controlled by controlling the working state of the air pump 310, thereby controlling the flow rate of the amplification reagent. Correspondingly, to fix the air pipe 320 on the top of the amplification reagent tube 200, an air pipe interface cover 330 is disposed at the end of the air pipe 320, and the air pipe interface cover 330 is detachably connected to the amplification reagent tube 200. In this embodiment, the air pipe interface cover 330 is threadedly connected to the amplification reagent tube 200, thereby ensuring the air tightness between the air pipe 320 and the amplification reagent tube 200 without affecting the detachable connection between the air pipe 320 and the amplification reagent tube 200. The air pipe 320 and the amplification reagent tube 200 may also be connected in other detachable methods. In addition, a filter material is placed between the air pipe interface cover 330 and the amplification reagent tube 200 to reduce the possibility of impurities entering the amplification reagent tube 200 with the airflow.

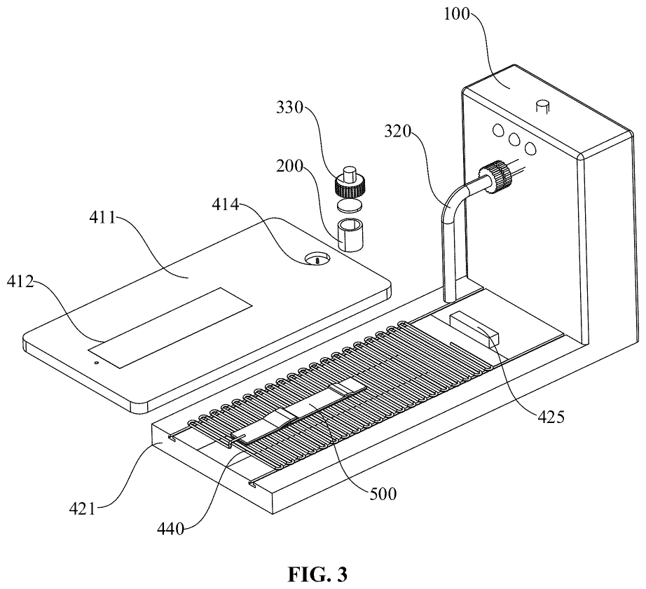

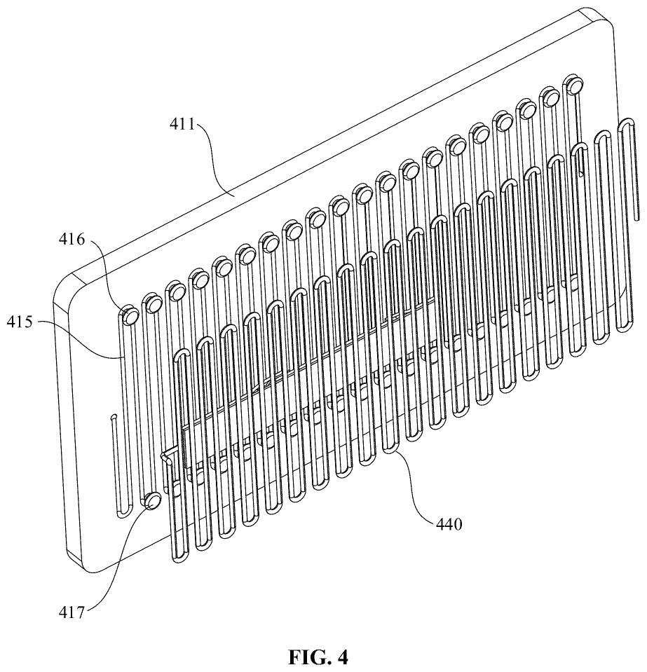

Referring to FIGS. 3 and 4, after the amplification reagent containing the to-be-tested nucleic acid sample enters the PCR pipeline 440, the heating device 420 heats the PCR pipeline 440 to complete the amplification. In an embodiment, the heating device 420 includes a base plate 421 and a heating assembly 430. The base plate 421 is disposed on a side of the body 100. The cover plate assembly 410 is disposed above the base plate 421. The heating assembly 430 is disposed on the base plate 421 and located between the cover plate assembly 410 and the base plate 421. The PCR pipeline 440 is disposed on the cover plate assembly 410 and also located between the cover plate assembly 410 and the base plate 421. The heating assembly 430 heats the PCR pipeline 440 to satisfy the temperature requirements for the three stages, reverse transcription, denaturation, and annealing and extension, during the PCR process. The amplification reagent moves in the PCR pipeline 440 and is heated by the heating assembly 430.

Referring to FIGS. 3 and 4, in this embodiment, the cover plate assembly 410 includes a cover plate 411, a first snap group 416, and a second snap group 417. The amplification reagent containing the to-be-tested nucleic acid sample flows in the PCR pipeline 440, and the first snap group 416 and the second snap group 417 fix the PCR pipeline 440 on the cover plate 411. In an embodiment, the first snap group 416 and the second snap group 417 each include multiple snaps. The first snap group 416 and the second snap group 417 are separately disposed on the two sides of the same surface of the cover plate 411, and the length direction of the first snap group 416 and the length direction of the second snap group 417 are both parallel to the length direction of the cover plate 411. The PCR pipeline 440 is wound around the snaps of the first snap group 416 and the snaps of the second snap group 417 in sequence, thus, the PCR pipeline 440 forms a serpentine shape. The first snap group 416 and the second snap group 417 fix the PCR pipeline 440, and a flume tank 415 into which the PCR pipeline 440 is embedded is further opened on the cover plate 411, thereby further improving the stability of the PCR pipeline 440. The PCR pipeline 440 forms the serpentine shape so that the length is increased, the PCR reaction time of the to-be-tested nucleic acid sample flowing in the PCR pipeline 440 is increased, the order of magnitude of the to-be-tested nucleic acid sample is increased, and subsequent detection is facilitated. It is to be noted that a test tube slot 414 into which the amplification reagent tube 200 is placed is opened on the cover plate 411, a first end of the PCR pipeline 440 is connected to the liquid outlet 210, that is, a tube slot into which the end of the PCR pipeline 440 is embedded is opened at the bottom of the test tube slot 414, and an interface is further disposed on the tube slot to facilitate the connection between the PCR pipeline 440 and the liquid outlet 210. After the to-be-tested nucleic acid sample is amplified by the heating device 420, at the second end of the PCR pipeline 440, the amplification product of the to-be-tested nucleic acid sample drops directly onto the test strip 500 for interpretation.

Referring to FIGS. 4 and 5, in the embodiments of the present application, to heat the PCR pipeline 440, that is, to complete the PCR amplification of the to-be-tested nucleic acid sample, a constant-temperature region 423, a high-temperature region 422, and a low-temperature region 424 are separately provided on a surface of the base plate 421 facing the cover plate 411. It is to be noted that the position corresponding to the constant-temperature region 423 is the corresponding position of the test tube slot 414, the high-temperature region 422 corresponds to the first snap group 416, and the low-temperature region 424 corresponds to the second snap group 417. The constant-temperature region 423 first heats the amplification reagent tube 200 so that reverse transcription of the to-be-tested nucleic acid sample occurs in the amplification reagent tube 200. After a certain period of time, the air supply device 300 pushes the amplification reagent containing the to-be-tested nucleic acid sample into the PCR pipeline 440. Pushed by the air supply device 300, the to-be-tested nucleic acid sample first passes through the high-temperature region 422 and then reaches the low-temperature region 424 to complete denaturation as well as annealing and extension. Then, the to-be-tested nucleic acid sample in the PCR pipeline 440 continues passing through the high-temperature region 422 and the low-temperature region 424 and thus is continuously amplified. When the amplification product of the to-be-tested nucleic acid sample reaches the test strip 500 through the PCR pipeline 440, the order of magnitude has increased so that it is easy to use the test strip 500 to interpret the to-be-tested nucleic acid sample.

Referring to FIG. 5, in an embodiment, the heating assembly 430 includes three heating parts. The three heating parts are separately a low-temperature part 431, a high-temperature part 432, and a constant-temperature part 433. The constant-temperature part 433 is disposed in the constant-temperature region 423, and the temperature is set at 52° C. to 55° C. The high-temperature part 432 is disposed in the high-temperature region 422, and the temperature is set at 97° C. to 99° C. The low-temperature part 431 is disposed in the low-temperature region 424, and the temperature is set at 57° C. to 59° C. In the embodiment of the present application, the low-temperature part 431, the high-temperature part 432, and the constant-temperature part 433 all use heating films. In addition to reaching the set temperature quickly, the heating films facilitate the control of temperature change. In addition, the setting of the heating films reduces the gap between the cover plate 411 and the base plate 421 so that the PCR pipeline 440 can fit the base plate 421 as much as possible, thereby making the PCR pipeline 440 more susceptible to heat and ensuring the heating effect of the heating assembly 430 on the PCR pipeline 440, that is, ensuring the amplification of the to-be-tested nucleic acid sample.

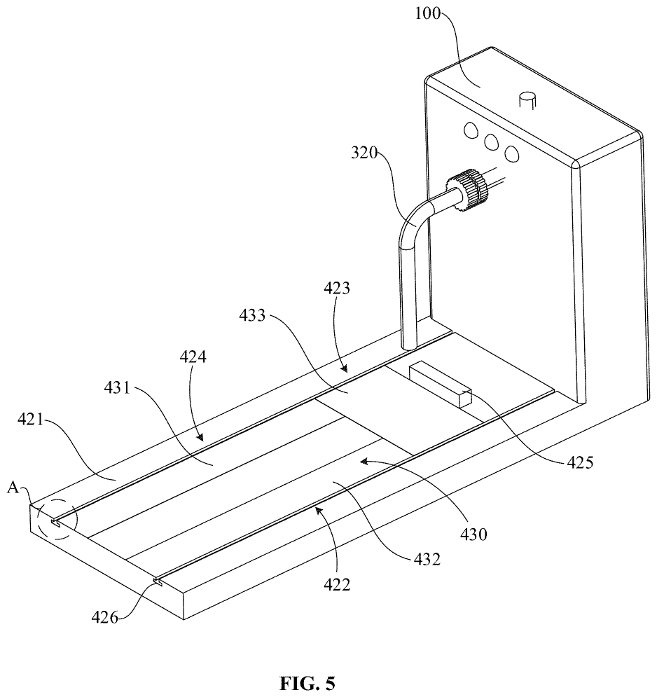

Referring to FIGS. 3 and 6, in an embodiment, to ensure the heating effect, that is, to ensure the fixed relationship between the cover plate 411 and the base plate 421, T-slots 426 into which the first snap group 416 and the second snap group 417 are embedded are opened on the base plate 421. It is to be noted that an end of the T-slot 426 facing away from the body 100 penetrates through the base plate 421. During an actual installation or disassembly process, the cover plate 411 slides from an end of the base plate 421 facing away from the body 100 to the other end. During sliding, the heads of the snaps are embedded into the T-slots 426, and the slot opening 427 of the T-slot 426 limits the heads of the snaps and prevents the snaps from moving out of the T-slots 426, thereby fixing the cover plate 411 on the base plate 421. To ensure the correspondence between the constant-temperature region 423 and the test tube slot 414, that is, to ensure the heating effect of the heating assembly 430 on the PCR pipeline 440, a limiting block 425 is further disposed on the base plate 421. The limiting block 425 is provided to limit the range within which the cover plate 411 is movable on the base plate 421. During the installation, the accurate installation position of the cover plate 411 can be ensured just by pushing the cover plate 411 until the cover plate 411 abuts against the limiting block 425.

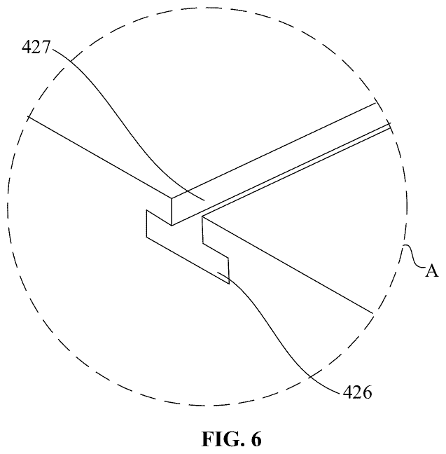

Referring to FIG. 7, in the embodiments of the present application, a window groove 412 is opened on the cover plate 411, and a test strip slot 413 is further opened at the bottom of the window groove 412. The test strip 500 is embedded into the test strip slot 413, and an end of the PCR pipeline 440 extends to the test strip 500 so that the amplified to-be-tested nucleic acid sample drops onto the test strip 500. An observation window 600 may be mounted in the window groove 412 and may be made of transparent materials such as glass and resin, thereby facilitating the result interpretation. The test strip 500 may be mounted on the observation window 600.

The usage examples of the present application are described below.

When nucleic acid screening is performed, the to-be-tested nucleic acid sample is put into the amplification reagent tube 200, and at the same time, the test strip 500 is put into the test strip slot 413. Then, the air supply device 300 and the heating device 420 are started through the switch on the body 100. The air pump 310 works to generate air pressure in the amplification reagent tube 200 to push the to-be-tested nucleic acid sample into the PCR pipeline 440. The heating assembly 430 heats the PCR pipeline 440, and the amplification of the to-be-tested nucleic acid sample is completed in the PCR pipeline 440. The amplified to-be-tested nucleic acid sample drops onto the test strip 500, and then the detection result of the amplified to-be-tested nucleic acid sample is interpreted through the test strip 500.

In conjunction with the preceding usage process, further explanation is made below in conjunction with the principles of the design of the present application.

The present application further proposes a chromatography-based multiplex nucleic acid detection method. In the present application, a chromatography test strip is used to detect the amplification product of to-be-tested deoxyribonucleic acid (DNA)/ribonucleic acid (RNA). In an embodiment, based on the technical principles of PCR temperature-changing amplification and lateral chromatography, up to 13 target sequences are amplified simultaneously, and then a lateral chromatography test strip is used for detection and interpretation.

The multiplex nucleic acid detection method proposed in the present application includes the steps described below.

In S1, a to-be-tested nucleic acid sample is added to a multiplex PCR amplification reagent set.

In S2, temperature-changing amplification is performed on the multiplex PCR amplification reagent set.

In S3, an amplification product is detected by the test strip and a result is read from the test strip.

The multiplex PCR amplification reagent set includes a mixed enzyme, a PCR reaction system, and specific primers.

The mixed enzyme includes reverse transcriptase, DNA polymerase, and uracil-N-glycosylase (UNG). The reverse transcriptase is Moloney murine leukemia virus (M-MLV) reverse transcriptase, and the DNA polymerase is Thermusaquaticus (Taq) DNA polymerase.

The PCR reaction system includes a ribonuclease inhibitor, dNTP (2′-deoxyuridine 5′-triphosphate, dUTP), Tris-HCL, KCL, and MgCl₂.

The specific primers are a first primer and a second primer that are designed according to different target sequences and have a specific amplifiable length of 100 bp to 150 bp.

It is to be noted that the main function of the mixed enzyme is to reverse-transcribe the to-be-tested RNA into cDNA and complete DNA replication; the PCR reaction system provides an optimal catalytic reaction condition for the enzyme; the first primer and the second primer for multiplex target amplification can specifically bind the target DNA/RNA in the to-be-tested sample; further, the first primer is labeled with a marker, and the second primer is labeled with a nucleic acid sequence unrelated to the target sequence and sample.

In an embodiment, the marker is biotin, carboxyfluorescein (FAM), green fluorescent protein (VIC), fluorescein isothiocyanate (FITC), digoxin, or other common modification markers of the primer; a marker sequence is a nucleic acid sequence that is unrelated to the target sequence and sample, and different sequences are separately labeled with different targets, that are recorded as B1, B2, B3, . . . in the following description of the present application. Performing the temperature-changing amplification on the multiplex PCR amplification reagent set includes the steps described below.

In A1, reverse transcription is performed on the multiplex PCR amplification reagent set in the constant-temperature region with a temperature range of 52° C. to 55° C.

In A2, the multiplex PCR amplification reagent set is denatured in the high-temperature region with a temperature range of 97° C. to 99° C.

In A3, annealing and extension are performed on the multiplex PCR amplification reagent set in the low-temperature region with a temperature range of 57° C. to 59° C.

In A4, steps A2 and A3 are repeated several times.

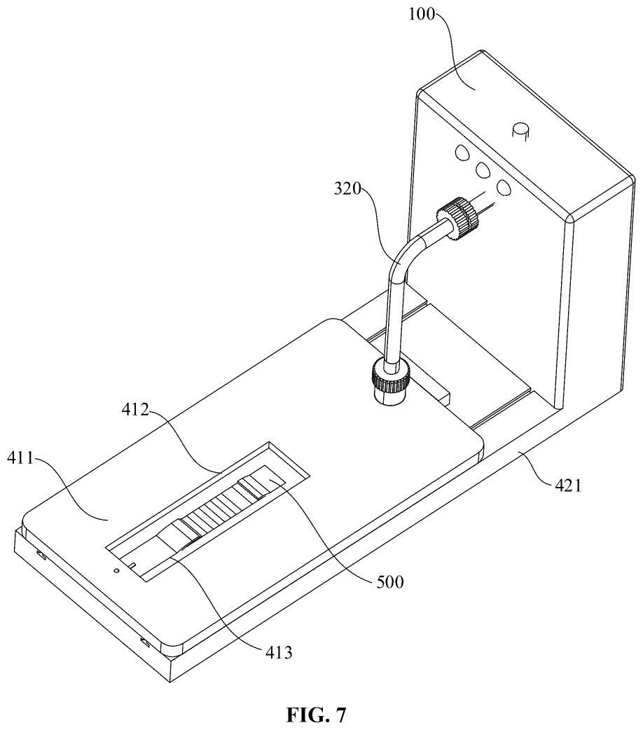

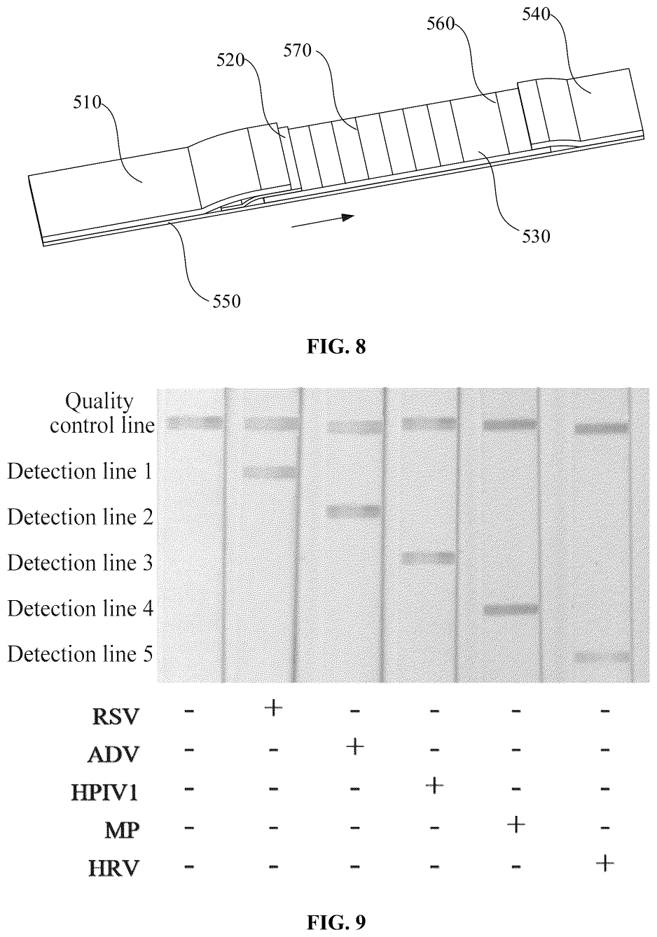

In the present application, the structure of the test strip 500 is shown in FIG. 8 and includes a sample pad 510, a marker pad 520, a capture membrane 530, a water absorbent pad 540, and a support base plate 550. The capture membrane 530 contains a combination of line C 560 and line T 570.

The sample pad 510 is used for carrying a sample solution, and has a certain filtering and buffering effect on the to-be-tested sample to reduce the interference of the ionic strength or pH in the sample on the detection result. The marker pad 520 is a binding chromogen labeled with a chromogenic substance. It is to be noted that the chromogenic substance may be colored microspheres; and the binding chromogen may bind to biotin or other markers with which the first primer in the sample is labeled. The capture membrane 530 carries the nucleic acid capture probes and is an important region where the hybridization reaction occurs. The pre-coated nucleic acid capture probes are a detection line (line T 570) and a quality control line (line C 560) and can generate complementary base pairing with B1, B2, B3, . . . with which the second primer is labeled so that the binding chromogen accumulates at the detection line, and the result is interpreted according to the color development of the detection line. The water absorbent pad 540 provides the power of chromatography. The liquid sample flows upward through the water absorption of the water absorbent pad 540 and the capillary action of the capture membrane 530 so that the nucleic acid sample in the sample pad 510 is driven to move upward and react with the nucleic acid capture probe at the detection line. The support base plate 550 supports the entire test strip.

It is to be noted that the sample pad 510 is glass fiber, filter paper, or a blood filtration membrane, and the marker pad 520 is glass fiber, filter paper, or a polyester film. When the sample pad 510 and the marker pad 520 are made of the same material, the sample pad 510 and the marker pad 520 can be integrated so that a piece of glass fiber, filter paper, or polyester film is used to implement the functions of the sample pad 510 and the marker pad 520. In an embodiment, the binding chromogen labeled by the chromogenic substance on the marker pad 520 is streptavidin (SA), FAM, VIC, FITC, or the monoclonal antibody corresponding to digoxin. The capture membrane 530 may be a nitrocellulose (NC) membrane, a nylon membrane, or the like. Line T 570 contains the nucleic acid capture probe. Line C 560 contains the positive control nucleic acid capture probe. The coating concentration of the nucleic acid capture probe is 10 μM to 30 μM, the sequence length range is 20 bp to 45 bp, and a random sequence of 10 bp to 25 bp is added to each of the two ends. The water absorbent pad 540 is made of nonwoven material, and the support base plate 550 is a plastic plate.

As shown in FIG. 8, the working process is as follows: when the sample solution is added to the sample pad 510 of the test strip, the liquid moves along the direction of the horizontal arrow and first reaches the marker pad 520, and the marker with which the first primer in the sample is labeled binds to the binding chromogen labeled with the chromogenic substance (such as the colored microspheres) in the marker pad 520 to form a complex, that is, ‘marker-binding chromogen-colored microsphere’. The solution continues moving to reach line T/C of the capture membrane 530, the marker sequence with which the second primer is labeled and the nucleic acid capture probe here form a ‘nucleic acid capture probe-marker sequence’ complex, and the complex is fixed on line T/C. The solution continues moving forward and is absorbed by the water absorbent pad 540 at the end of the test strip. Finally, the result is determined according to the color development of the detection line and quality control line.

When a to-be-tested nucleic acid fragment exists, a ‘nucleic acid capture probe-marker sequence-nucleic acid sample-marker-binding chromogen-colored microsphere’ complex is formed and stays at line T 570, forming a colored strip visible to naked eyes, which indicates positive.

When the to-be-tested nucleic acid fragment does not exist, no ‘nucleic acid capture probe-marker sequence-nucleic acid sample-marker-binding chromogen-colored microsphere’ complex is formed, and the colored microspheres cannot accumulate at line T 570, so no colored strip visible to naked eyes is formed, which indicates negative.

Regardless of whether the to-be-tested nucleic acid fragment exists, a ‘nucleic acid capture probe-marker sequence-positive control nucleic acid-marker-binding chromogen-colored microsphere’ complex is formed and accumulates at line C 560, forming a colored strip visible to naked eyes, which indicates a valid experimental result. If the quality control line does not develop color, the test strip is invalid.

Based on the preceding chromatography-based multiplex nucleic acid detector, the present application is described below in conjunction with the embodiments and comparative examples.

In embodiment one, the coating concentration of the nucleic acid capture probe of the chromatography-based multiplex nucleic acid detection test strip is 25 μM, the sequence length of the nucleic acid capture probe is 35 bp, and a random sequence of 15 bp is added to each end.

In the embodiment, the DNA/RNA amplification template, the first primer labeled with the marker, the second primer labeled with the marker sequence, the nucleic acid capture probe, and the binding chromogen are synthesized by a biotechnology company, and in this embodiment, the marker is biotin, and the binding chromogen is SA labeled with the colored microspheres.

In this embodiment, the chromatography-based multiplex nucleic acid detection test strip is used to detect items including respiratory syncytial virus (RSV), adenovirus (ADV), human parainfluenza virus 1 (HPIV1), Mycoplasma pneumoniae (MP), human rhinovirus (HRV), and ribonucleoprotein (RNP).

The preparation of the chromatography-based multiplex nucleic acid detection test strip includes the steps described below.

1) The colored microsphere-labeled SA solution is sprayed onto the marker pad 520 by a film sprayer at a sprayer speed of 1 μL/cm, and the marker pad onto which the solution is sprayed is placed in a 37° C. incubator and dried for 1 hour. The portion of the marker pad 520 containing colored microsphere-labeled SA is cut to obtain a strip of 5 mm wide and 30 cm long.

2) The film sprayer is used to streak the nucleic acid capture probe and the positive control nucleic acid probe on the capture membrane 530 at a streaking speed of 0.6 μL/cm, and the streaked capture membrane 530 is placed in a 37° C. incubator and dried for 16 hours.

3) According to the structure of the test strip in FIG. 8, the capture membrane 530 is pasted on the support base plate 550, the marker pad 520 and the sample pad 510 are pasted in sequence on a side close to line T 570, the water absorbent pad 540 is pasted on a side close to line C 560, a test strip of 3.08 mm wide is cut through a cutter, and the test strip is sealed and stored in an aluminum foil bag for later use.

Detection is performed according to the detection principle of the chromatography-based multiplex nucleic acid detection method. (1) The target plasmid is used as a template to perform multiplex gene amplification. (2) The multiplex nucleic acid amplification product drops onto the sample pad 510 of an elongated chromatography test strip, the multiplex nucleic acid amplification product flows from the sample pad 510 to the colored-microsphere marker pad 520 to specifically bind a colored microsphere-labeled conjugate on the marker pad 520, then flows through the detection line and the quality control line on the capture membrane 530, and finally, flows to the water absorbent pad 540. The color of the detection line and the color of the quality control line are observed to determine the detection result.

In embodiment two, the coating concentration of the nucleic acid capture probe of the chromatography-based multiplex nucleic acid detection test strip is 10 μM, the sequence length is 35 bp, a random sequence of 15 bp is added to each end of the sequence, and the remaining settings are the same as those in embodiment one.

In embodiment three, the coating concentration of the nucleic acid capture probe of the chromatography-based multiplex nucleic acid detection test strip is 30 μM, the sequence length is 35 bp, a random sequence of 15 bp is added to each end of the sequence, and the remaining settings are the same as those in embodiment one.

In embodiment four, the coating concentration of the nucleic acid capture probe of the chromatography-based multiplex nucleic acid detection test strip is 25 μM, the sequence length is 45 bp, a random sequence of 15 bp is added to each end of the sequence, and the remaining settings are the same as those in embodiment one.

In embodiment five, the coating concentration of the nucleic acid capture probe of the chromatography-based multiplex nucleic acid detection test strip is 25 μM, the sequence length is 20 bp, a random sequence of 15 bp is added to each end of the sequence, and the remaining settings are the same as those in embodiment one.

In embodiment six, the coating concentration of the nucleic acid capture probe of the chromatography-based multiplex nucleic acid detection test strip is 25 μM, the sequence length is 35 bp, a random sequence of 10 bp is added to each end of the sequence, and the remaining settings are the same as those in embodiment one.

In embodiment seven, the coating concentration of the nucleic acid capture probe of the chromatography-based multiplex nucleic acid detection test strip is 25 μM, the sequence length is 35 bp, a random sequence of 25 bp is added to each end of the sequence, and the remaining settings are the same as those in embodiment one.

In embodiment eight, for the three regions of the temperature-changing PCR, the temperature of the constant-temperature region is 52° C., the temperature of the high-temperature region is 98° C., the temperature of the low-temperature region is 58° C., and the remaining settings are the same as those in embodiment one.

In embodiment nine, for the three regions of the temperature-changing PCR, the temperature of the constant-temperature region is 52° C., the temperature of the high-temperature region is 97° C., the temperature of the low-temperature region is 59° C., and the remaining settings are the same as those in embodiment one.

In embodiment ten, for the three regions of the temperature-changing PCR, the temperature of the constant-temperature region is 54° C., the temperature of the high-temperature region is 97° C., the temperature of the low-temperature region is 57° C., and the remaining settings are the same as those in embodiment one.

In embodiment eleven, for the three regions of the temperature-changing PCR, the temperature of the constant-temperature region is 55° C., the temperature of the high-temperature region is 99° C., the temperature of the low-temperature region is 58° C., and the remaining settings are the same as those in embodiment one.

In embodiment twelve, for the three regions of the temperature-changing PCR, the temperature of the constant-temperature region is 55° C., the temperature of the high-temperature region is 97° C., the temperature of the low-temperature region is 59° C., and the remaining settings are the same as those in embodiment one.

In comparative example one, the coating concentration of the nucleic acid capture probe of the chromatography-based multiplex nucleic acid detection test strip is 8 μM, the sequence length is 35 bp, a random sequence of 15 bp is added to each end of the sequence, and the remaining settings are the same as those in embodiment one.

In comparative example two, the coating concentration of the nucleic acid capture probe of the chromatography-based multiplex nucleic acid detection test strip is 35 μM, the sequence length is 35 bp, a random sequence of 15 bp is added to each end of the sequence, and the remaining settings are the same as those in embodiment one.

In comparative example three, the coating concentration of the nucleic acid capture probe of the chromatography-based multiplex nucleic acid detection test strip is 25 μM, the sequence length is 15 bp, a random sequence of 15 bp is added to each end of the sequence, and the remaining settings are the same as those in embodiment one.

In comparative example four, the coating concentration of the nucleic acid capture probe of the chromatography-based multiplex nucleic acid detection test strip is 25 μM, the sequence length is 50 bp, a random sequence of 15 bp is added to each end of the sequence, and the remaining settings are the same as those in embodiment one.

In comparative example five, the coating concentration of the nucleic acid capture probe of the chromatography-based multiplex nucleic acid detection test strip is 25 μM, the sequence length is 35 bp, a random sequence of 7 bp is added to each end of the sequence, and the remaining settings are the same as those in embodiment one.

In comparative example six, the coating concentration of the nucleic acid capture probe of the chromatography-based multiplex nucleic acid detection test strip is 25 μM, the sequence length is 35 bp, a random sequence of 28 bp is added to each end of the sequence, and the remaining settings are the same as those in embodiment one.

In comparative example seven, for the three regions of the temperature-changing PCR, the temperature of the constant-temperature region is 51° C., the temperature of the high-temperature region is 98° C., the temperature of the low-temperature region is 58° C., and the remaining settings are the same as those in embodiment one.

In comparative example eight, for the three regions of the temperature-changing PCR, the temperature of the constant-temperature region is 51° C., the temperature of the high-temperature region is 97° C., the temperature of the low-temperature region is 59° C., and the remaining settings are the same as those in embodiment one.

In comparative example nine, for the three regions of the temperature-changing PCR, the temperature of the constant-temperature region is 52° C., the temperature of the high-temperature region is 96° C., the temperature of the low-temperature region is 58° C., and the remaining settings are the same as those in embodiment one.

In comparative example ten, for the three regions of the temperature-changing PCR, the temperature of the constant-temperature region is 52° C., the temperature of the high-temperature region is 100° C., the temperature of the low-temperature region is 57° C., and the remaining settings are the same as those in embodiment one.

In comparative example eleven, for the three regions of the temperature-changing PCR, the temperature of the constant-temperature region is 54° C., the temperature of the high-temperature region is 98° C., the temperature of the low-temperature region is 56° C., and the remaining settings are the same as those in embodiment one.

In comparative example twelve, for the three regions of the temperature-changing PCR, the temperature of the constant-temperature region is 54° C., the temperature of the high-temperature region is 97° C., the temperature of the low-temperature region is 60° C., and the remaining settings are the same as those in embodiment one.

### Performance Detection

### 1. Color Development Testing of Chromatography-Based Multiplex Nucleic Acid

To test the chromatography color development effect under different coating concentrations and sequence lengths of the nucleic acid capture probe, and different lengths added to the two ends of the sequence, the same amplified sample is used for chromatography color development comparison testing.

The detection method is as follows: RNP (the template plasmid concentration is 100 copies/μL) is amplified as a unified sample, RNP nucleic acid capture probe chromatography test strips with different conditions are prepared, the chromatography color development effect of the unified sample is tested using the chromatography test strips with different conditions, three repeated tests are performed for each condition, and the color development degrees are recorded.

Detection results are described below.

TABLE 1
Chromatography color development results
in embodiments one to seven

| | |
|---|---|
| Group | Color development degree |

| | | | |
|---|---|---|---|
| Embodiment one | +++ | +++ | +++ |
| Embodiment two | +++ | ++ | +++ |
| Embodiment three | ++ | +++ | +++ |
| Embodiment four | +++ | +++ | ++ |
| Embodiment five | +++ | ++ | +++ |
| Embodiment six | +++ | ++ | ++ |
| Embodiment seven | +++ | ++ | +++ |

Note:
The color development degree is divided into four categories: the color development is very clear (+++), the color development is relatively clear (++), the color development is relatively weak (+), and no color development (−).

According to Table 1, it can be seen that the chromatography color development effects in embodiments one to seven are relatively clear or very clear, indicating that the multiplex nucleic acid chromatography detection effect of the present application is good. The color development effect in embodiment one is very clear for the three repeated tests, indicating that the chromatography color development effect in embodiment one is significantly better than other embodiments.

TABLE 2
Chromatography color development results in embodiment
one and comparative examples one to six

| | |
|---|---|
| Group | Color development degree |

| | | | |
|---|---|---|---|
| Embodiment one | +++ | +++ | +++ |
| Comparative | + | + | − |
| example one | | | |
| Comparative | + | − | + |
| example two | | | |
| Comparative | − | − | + |
| example three | | | |
| Comparative | − | + | − |
| example four | | | |
| Comparative | + | − | + |
| example five | | | |
| Comparative | + | + | + |
| example six | | | |

Note:
The color development degree is divided into four categories: the color development is very clear (+++), the color development is relatively clear (++), the color development is relatively weak (+), and no color development (−).

According to Table 2, it can be seen that the chromatography color development effect in embodiment is very clear for the three repeated tests, while the chromatography color development effects in comparative examples one to six are relatively weak or no color development, indicating that the multiplex nucleic acid chromatography detection effect of the present application is good.

### 2. Specificity Testing

To verify the specificity of this method, whether different detection lines can specifically detect the corresponding samples is tested. That is, when a to-be-tested sample exists, the quality control line develops color and the corresponding detection line develops color, otherwise only the quality control line develops color.

The detection method is as follows: a 6-layer chromatography test strip for RSV, ADV, HPIV1, MP, HRV, and RNP is prepared according to the method in embodiment one. 0/1/2/3/4/5 target genes (all with RNP) are separately amplified, and the amplification product is tested using the chromatography test strip to verify the detection specificity.

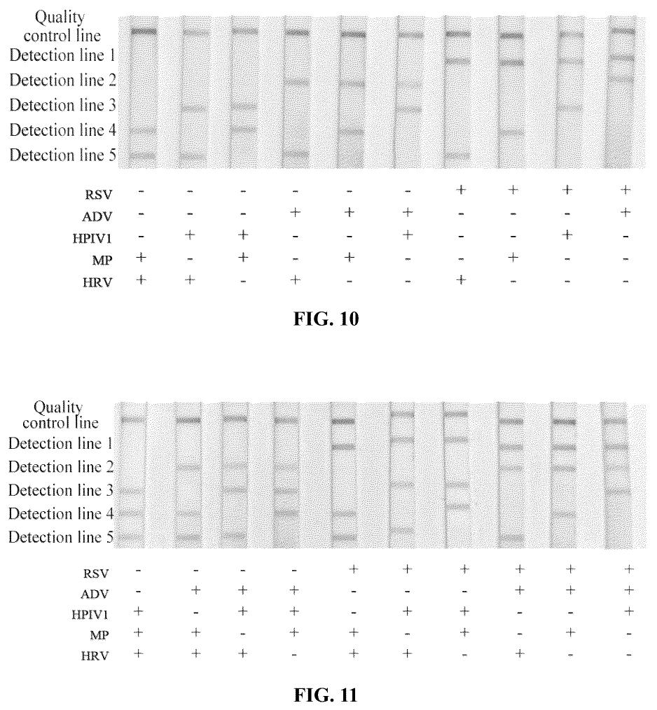

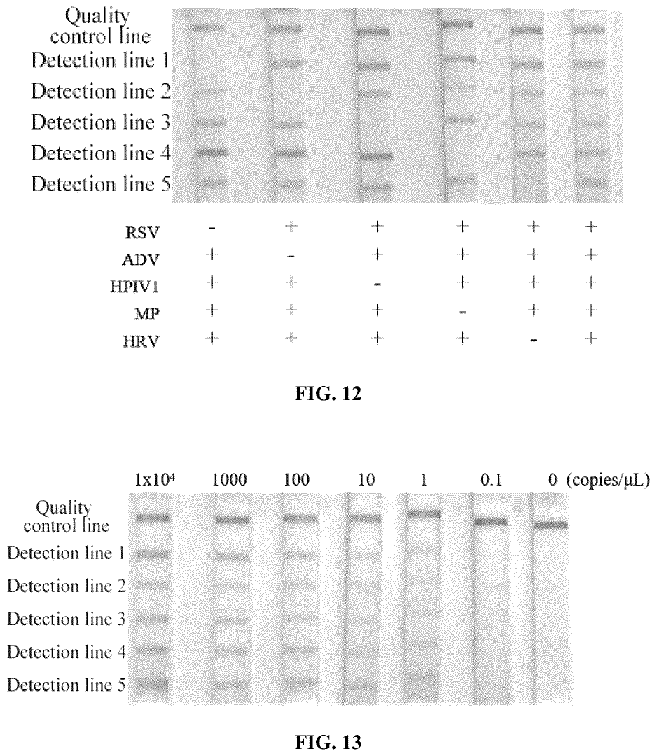

Referring to FIGS. 9 to 12, the results show that if the amplification product contains the five types of detected target DNA/RNA, five detection lines and the quality control line all develop color; if the amplification product contains only part of the five types of target DNA/RNA, only the corresponding detection lines and the quality control line develop color; and if the amplification product does not contain the five types of target DNA/RNA, the five detection lines do not develop color and only the quality control line develops color. It indicates that the multiplex nucleic acid detection method provided in the present application has strong specificity in detecting the target DNA/RNA.

### 3. Sensitivity Testing

To verify the sensitivity of the multiplex nucleic acid detection method provided in the present application, the lowest detection limit of this method is tested using the plasmids containing target genes at the calibrated concentration.

The detection method is as follows: the plasmids containing target genes at the calibrated concentration are used to perform the 10-fold concentration gradient dilution, each gradient is repeated three times, and the lowest dilution concentration with a 100% positive detection rate is used as an estimated detection limit. After the estimated detection limit is determined, the plasmids are diluted to a value near the estimated detection limit concentration, detection is performed using the chromatography test strip prepared in the manner of embodiment one, and each concentration value is tested 20 times to further accurately determine the lowest detection limit concentration (the dilution with a positive rate above 95% is selected as the lowest detection limit of this method).

Referring to FIG. 13, the results show that the lowest detection limit of the multiplex nucleic acid detection method provided in the present application is 1 copies/uL.

### 4. Stability Testing

To test the stability of this method, an acceleration stability test is adopted.

The detection method is as follows: the test strips of the same batch are separately placed in a 45° C. oven and a 55° C. oven, and placed at 45° C. for 90 days and at 55° C. for 45 days for detection, to test the sensitivity and specificity of the test strips.

TABLE 3
Sensitivity and specificity in embodiments one to seven and comparative
examples one to six after high-temperature acceleration

| | 45° C. for 90 days | | 55° C. for 45 days | |
|---|---|---|---|---|
| Condition | Sensitivity | Specificity | Sensitivity | Specificity |
| Embodiment | The lowest | All satisfy the | The lowest | All satisfy the |
| one | detection limit | requirements | detection limit | requirements |
| Embodiment | remains | | remains | |
| two | unchanged | | unchanged | |
| Embodiment | | | | |
| three | | | | |
| Embodiment | | | | |
| four | | | | |
| Embodiment | | | | |
| five | | | | |
| Embodiment | | | | |
| six | | | | |
| Embodiment | | | | |
| seven | | | | |
| Comparative | The lowest | | The lowest | |
| example one | detection limit | | detection limit | |
| Comparative | decreases | | decreases | |
| example two | | | | |
| Comparative | | | | |
| example | | | | |
| three | | | | |
| Comparative | | | | |
| example four | | | | |
| Comparative | | | | |
| example five | | | | |
| Comparative | | | | |
| example six | | | | |

The results show that when the chromatography-based multiplex nucleic acid detection test strip of the present application is placed at 45° C. for 90 days and 55° C. for 45 days, samples with the lowest detection limit concentration can still be detected, the specificity satisfies the requirements, and the product performance remains stable.

### 5. PCR Temperature-Changing Testing

To test the temperature setting range during the PCR temperature changing process in the multiplex nucleic acid detection method provided in the present application, the same template sample is used to perform temperature-changing amplification and detect the amplification product.

The detection method is as follows: RNP (the template plasmid concentration is 100 copies/μL) is used as a unified sample, the temperature setting is separately performed on the chips in the constant-temperature region, the high-temperature region, and the low-temperature region, the chromatography color development effect of the amplification product is tested using the test strips of the same batch, three repeated tests are performed for each condition, and the color development degrees are recorded.

TABLE 4
Chromatography color development results
in embodiments eight to twelve

| | |
|---|---|
| Group | Color development degree |

| | | | |
|---|---|---|---|
| Embodiment eight | +++ | +++ | +++ |
| Embodiment nine | ++ | +++ | +++ |
| Embodiment ten | +++ | +++ | ++ |
| Embodiment | +++ | +++ | ++ |
| eleven | | | |
| Embodiment | +++ | ++ | +++ |
| twelve | | | |

Note:
The color development degree is divided into four categories: the color development is very clear (+++), the color development is relatively clear (++), the color development is relatively weak (+), and no color development (−).

According to Table 4, it can be seen that the chromatography color development effects in embodiments eight to twelve are relatively clear or very clear, indicating that the temperature setting range during the PCR temperature changing process of the present application is reasonable and the detection effect is good. The color development effect in embodiment eight is very clear for the three repeated tests, indicating that the temperature setting amplification effect in embodiment eight is significantly better than that in other embodiments.

TABLE 5
Chromatography color development results in embodiment
eight and comparative examples seven to twelve

| | |
|---|---|
| Group | Color development degree |

| | | | |
|---|---|---|---|
| Embodiment eight | +++ | +++ | +++ |
| Comparative | − | + | − |
| example seven | | | |
| Comparative | + | − | + |
| example eight | | | |
| Comparative | − | + | + |
| example nine | | | |
| Comparative | + | + | − |
| example ten | | | |
| Comparative | + | − | − |
| example eleven | | | |
| Comparative | + | − | + |
| example twelve | | | |

Note:
The color development degree is divided into four categories: the color development is very clear (+++), the color development is relatively clear (++), the color development is relatively weak (+), and no color development (−).

According to Table 5, it can be seen that the chromatography color development effect in embodiment eight is very clear for the three repeated tests, while the chromatography color development effects in comparative examples seven to twelve are relatively weak or no color development, indicating that the temperature setting range during the PCR temperature changing process of the present application is reasonable and the detection effect is good.

1. A chromatography-based multiplex nucleic acid detector, comprising:
an air supply device, wherein an air outlet of the air supply device is configured to communicate with a first end of an amplification reagent tube;
a heating device;
a polymerase chain reaction (PCR) pipeline; and
a cover plate assembly mounted on the heating device, wherein an observation window is formed on the cover plate assembly, a PCR pipeline installation position is formed on at least one of the cover plate assembly or the heating device, the PCR pipeline is mounted at the PCR pipeline installation position, the heating device is configured to heat the PCR pipeline, and a test strip is mounted on the observation window;
wherein a second end of the amplification reagent tube communicates with a first end of the PCR pipeline, and a second end of the PCR pipeline is configured to output an amplified sample to the test strip.

2. The chromatography-based multiplex nucleic acid detector of claim 1, wherein the heating device comprises a base plate and a heating assembly, the cover plate assembly is disposed on the base plate, and the heating assembly is disposed between the cover plate assembly and the base plate.

3. The chromatography-based multiplex nucleic acid detector of claim 2, wherein the heating assembly comprises a high-temperature part, a low-temperature part, and a constant-temperature part, wherein the high-temperature part, the low-temperature part, and the constant-temperature part are disposed on the base plate and configured to simultaneously heat the PCR pipeline at different temperatures.

4. The chromatography-based multiplex nucleic acid detector of claim 2, wherein the cover plate assembly comprises a cover plate, a first snap group, and a second snap group, wherein the cover plate is detachably connected to the base plate, each of the first snap group and the second snap group has a plurality of snaps, the first snap group and the second snap group are separately disposed on two sides of a surface of the cover plate facing the base plate, a length direction of the first snap group is parallel to a length direction of the second snap group, and the PCR pipeline is wound around the first snap group and the second snap group in sequence and forms a serpentine shape.

5. The chromatography-based multiplex nucleic acid detector of claim 4, wherein two T-slots are opened on a surface of the base plate facing the cover plate, length directions of the two T-slots are parallel to each other, the first snap group and the second snap group are separately embedded into the two T-slots, the base plate is further provided with a limiting block configured to limit a position of the cover plate.

6. The chromatography-based multiplex nucleic acid detector of claim 4, wherein the observation window is opened on a side surface of the cover plate facing away from the base plate, a test tube slot for accommodating the amplification reagent tube is further opened on the side surface of the cover plate facing away from the base plate, and the PCR pipeline installation position is formed on a side surface of the cover plate facing the base plate.

7. The chromatography-based multiplex nucleic acid detector of claim 1, further comprising a body, wherein the heating device is disposed on the body, the air supply device comprises an air pump and an air pipe, the air pump is disposed inside the body, and two ends of the air pipe are separately connected to the air pump and the amplification reagent tube.

8. The chromatography-based multiplex nucleic acid detector of claim 7, wherein an air pipe interface cover is disposed at an end of the air pipe connected to the amplification reagent tube, and the air pipe interface cover is detachably connected to the amplification reagent tube.

9. A chromatography-based multiplex nucleic acid detection test strip, applied to a multiplex nucleic acid detector, wherein the test strip comprises a sample pad, a marker pad, a capture membrane, a water absorbent pad, and a support base plate, wherein the sample pad, the marker pad, the capture membrane, and the water absorbent pad are disposed on the support base plate in sequence;
wherein the chromatography-based multiplex nucleic acid detector comprises an air supply device, a heating device, a polymerase chain reaction (PCR) pipeline, and a cover plate assembly mounted on the heating device; wherein an air outlet of the air supply device is configured to communicate with a first end of an amplification reagent tube, an observation window is formed on the cover plate assembly, a PCR pipeline installation position is formed on at least one of the cover plate assembly or the heating device, the PCR pipeline is mounted at the PCR pipeline installation position, the heating device is configured to heat the PCR pipeline, and the test strip is mounted on the observation window; wherein a second end of the amplification reagent tube communicates with a first end of the PCR pipeline, and a second end of the PCR pipeline is configured to output an amplified sample to the test strip.

10. A chromatography-based multiplex nucleic acid detection method, applied to a multiplex nucleic acid detector and a multiplex nucleic acid detection test strip;
wherein the test strip comprises a sample pad, a marker pad, a capture membrane, a water absorbent pad, and a support base plate, wherein the sample pad, the marker pad, the capture membrane, and the water absorbent pad are disposed on the support base plate in sequence; the chromatography-based multiplex nucleic acid detector comprises an air supply device, a heating device, a polymerase chain reaction (PCR) pipeline, and a cover plate assembly mounted on the heating device; wherein an air outlet of the air supply device is configured to communicate with a first end of an amplification reagent tube, an observation window is formed on the cover plate assembly, a PCR pipeline installation position is formed on at least one of the cover plate assembly or the heating device, the PCR pipeline is mounted at the PCR pipeline installation position, the heating device is configured to heat the PCR pipeline, and the test strip is mounted on the observation window; wherein a second end of the amplification reagent tube communicates with a first end of the PCR pipeline, and a second end of the PCR pipeline is configured to output an amplified sample to the test strip; and
the method comprises:
adding a to-be-tested nucleic acid sample to a multiplex PCR amplification reagent set;
performing temperature-changing amplification on the multiplex PCR amplification reagent set; and
detecting an amplification product by the test strip, and reading a result from the test strip.

11. The chromatography-based multiplex nucleic acid detection method of claim 10, wherein the multiplex PCR amplification reagent set comprises a mixed enzyme, a PCR reaction system, and a specific primer;
wherein the mixed enzyme comprises reverse transcriptase, deoxyribonucleic acid (DNA) polymerase, and uracil-N-glycosylase (UNG);
the PCR reaction system comprises a ribonuclease inhibitor, dNTP (dUTP), Tris-HCl, KCl, and MgCl₂; and
the specific primer comprises a first primer and a second primer, wherein the first primer is provided with a marker, and the second primer is provided with a marker sequence.

12. The chromatography-based multiplex nucleic acid detection method of claim 11, wherein the marker is one of biotin, carboxyfluorescein (FAM), green fluorescent protein (VIC), fluorescein isothiocyanate (FITC), or digoxin, a marker pad is disposed on the test strip, and a binding chromogen that binds to the marker is disposed on the marker pad.

13. The chromatography-based multiplex nucleic acid detection method of claim 11, wherein the marker sequence is a sequence independent of a target sequence and the sample, the test strip is provided with a line T and a line C, and each of the line T and the line C is provided with a nucleic acid capture probe for a specific binding with the marker sequence.

14. The chromatography-based multiplex nucleic acid detection method of claim 11, wherein a specific amplifiable length of each of the first primer and the second primer is 100 bp to 150 bp.

15. The chromatography-based multiplex nucleic acid detection method of claim 10, wherein performing the temperature-changing amplification on the multiplex PCR amplification reagent set comprises:
performing reverse transcription on the multiplex PCR amplification reagent set in a constant-temperature region;
denaturing the multiplex PCR amplification reagent set in a high-temperature region;
performing annealing and extension on the multiplex PCR amplification reagent set in a low-temperature region; and
sequentially repeating the following multiple times: denaturing the multiplex PCR amplification reagent set in the high-temperature region and performing annealing and extension on the multiplex PCR amplification reagent set in the low-temperature region;
wherein the constant-temperature region has a temperature range of 52° C. to 55° C., the high-temperature region has a temperature range of 97° C. to 99° C., and the low-temperature region has a temperature range of 57° C. to 59° C.

16. The chromatography-based multiplex nucleic acid detection test strip of claim 9, wherein the heating device comprises a base plate and a heating assembly, the cover plate assembly is disposed on the base plate, and the heating assembly is disposed between the cover plate assembly and the base plate.

17. The chromatography-based multiplex nucleic acid detection test strip of claim 16, wherein the heating assembly comprises a high-temperature part, a low-temperature part, and a constant-temperature part, wherein the high-temperature part, the low-temperature part, and the constant-temperature part are disposed on the base plate and configured to simultaneously heat the PCR pipeline at different temperatures.

18. The chromatography-based multiplex nucleic acid detection test strip of claim 16, wherein the cover plate assembly comprises a cover plate, a first snap group, and a second snap group, wherein the cover plate is detachably connected to the base plate, each of the first snap group and the second snap group has a plurality of snaps, the first snap group and the second snap group are separately disposed on two sides of a surface of the cover plate facing the base plate, a length direction of the first snap group is parallel to a length direction of the second snap group, and the PCR pipeline is wound around the first snap group and the second snap group in sequence and forms a serpentine shape.

19. The chromatography-based multiplex nucleic acid detection test strip of claim 18, wherein two T-slots are opened on a surface of the base plate facing the cover plate, length directions of the two T-slots are parallel to each other, the first snap group and the second snap group are separately embedded into the two T-slots, the base plate is further provided with a limiting block configured to limit a position of the cover plate.

20. The chromatography-based multiplex nucleic acid detection test strip of claim 18, wherein the observation window is opened on a side surface of the cover plate facing away from the base plate, a test tube slot for accommodating the amplification reagent tube is further opened on the side surface of the cover plate facing away from the base plate, and the PCR pipeline installation position is formed on a side surface of the cover plate facing the base plate.
